(19) United States
(12) Patent Application Publication
(10) Pub. No.: US 2012/0258892 A1
(43) Pub. Date: Oct. 11, 2012

(54) Methods, Compositions, and Kits for Making Targeted Nucleic Acid Libraries

(76) Inventor: Yan Wang, San Diego, CA (US)

(21) Appl. No.: 13/442,291

(22) Filed: Apr. 9, 2012

Related U.S. Application Data
(60) Provisional application No. 61/473,622, filed on Apr. 8, 2011.

(51) Int. Cl. C40B 50/18; C12N 9/12

(52) U.S. Cl. 506/32; 435/194

(57) ABSTRACT

The present invention provides a method and a kit for selecting and enriching target sequences specific for a genomic region of interest or a subset of a transcriptome using a target-capturing sequence library. The target-capturing sequence library comprises random DNA fragments generated from a target sequence template encompassing all the target sequences. The present invention provides an efficient and cost-effective method of target selection for targeted genome resequencing.

### CROSS-REFERENCE TO RELATED APPLICATIONS

This application claims the benefit of U.S. provisional patent application No. 61/473,622, filed Apr. 8, 2011, the contents of which are incorporated by reference herein.

### FIELD OF THE INVENTION

This invention relates to methods, compositions, and kits for making a DNA library preparation of a selected subset of a DNA/RNA sample. More specifically, it relates to methods for selecting and enriching target DNA/RNA sequences specific for regions of interest using a target-capturing sequence library.

### BACKGROUND OF THE INVENTION

Massive parallel sequencing technologies, also known as next generation sequencing (NGS), provide researchers with valuable genome-scale sequence information in an unparalleled throughput with the capacity of sequencing one whole human genome in two weeks. However, many researchers prefer to focus on certain portions of genome or transcriptome of interest, for example, a disease-related region, and screen through more samples. Targeted genome resequencing is a great way to reduce the sequencing cost per unit, while still provides uncompromised information for researchers to answer their specific questions.

One of the important bottlenecks for targeted genome resequencing is preparation of a DNA sample specific for a targeted region of a genome. Current technologies for targeted sample preparation fall into two categories. The first category uses oligonucleotide capture arrays specifically designed for genomic regions of interest. This technology requires design of up to hundreds of thousands of oligonucleotides to produce capture arrays. Researchers either need to purchase pre-made arrays or order their own custom arrays. In addition, the array-based preparation normally requires expensive instruments. The cost per reaction is very high. The second category involves use of polymerase chain reaction (PCR) to amplify DNA fragments of interest. Since a unique reaction is required for each fragment, the selection of large genomic regions requires the parallel design, optimization and execution of up to thousands of individual reactions. Cost on PCR primers and reagents can escalate. Mutations and bias introduced through PCR can often distort the results. Another disadvantage of these techniques is that both methods depend on known sequence information of targeted regions to design oligonucleotides or PCR primers. They are not applicable to preparation of sequencing samples for targeted genomic regions without substantial sequence information available.

As such, there is a great need in the art for technologies to make high quality DNA preparations of targeted genome regions in an efficient and cost-effective way. The present invention satisfies this need and provides other benefits as well.

### SUMMARY OF THE INVENTION

Circumventing the need of designing thousands of oligonucleotide probes or performing thousands of PCRs, the present invention enables researcher to make high quality DNA libraries specific for targeted genomic regions of interest, or a subset of transcriptome in an efficient and cost-effective way.

The present invention provides a method for enriching and selecting DNA/RNA sequences of a targeted subset sequences from a nucleic acid sample using a target-capturing DNA/RNA library. The target-capturing DNA library is generated by randomly fragmenting a target DNA template, for example, a BAC construct of a genomic region of interest, which ideally encompasses all the DNA sequences of interest. The pool of random DNA fragments generated from the target DNA template is linked to a capture domain to make a target-capturing DNA library. The target-capturing DNA library is then used to capture target sequences of interest from a population of nucleic acid sequences obtained from a DNA/RNA sample, for example, a DNA sample from a patient. The method of the invention can be applied for both genome and transcriptome target selection and enrichment. In addition to capture target sequences, the method of the invention can also be applied to remove target nucleic acids from a nucleic acid sample, for example, the target-capturing library can be used to remove high abundance house-keeping genes in a transcriptome. This invention can be used for gene discovery studies in finding disease-specific transcripts from a patient's sample.

The target sequences used herein refer to nucleic acid sequences containing sequences of interest. A target sequence can be a long stretch of genomic sequence, a cDNA sequence, or a short DNA fragment of the region of interest. The target sequences are particularly referred to a collection of short DNA sequences generated from a region of interest that needs to be sequenced. The target sequences are constituted of a subset of a whole population of nucleic acids, which need to be selected and/or enriched for further studies, e.g. high throughput sequencing. A target sequence template includes a continuous region of a DNA sequence (e.g. a BAC construct of a genomic region) or a collection of DNA sequences (e.g. PCR products of genes of interest, or cDNA sequences) or DNAs extracted from special sources (e.g. DNAs from CHIP (chromatin immunoprecipitation) assays) that ideally encompasses all the sequences of target sequences. A target sequence template can also be RNA sequences, for example, rRNAs, mRNAs, siRNAs, snRNA, or RNAs extracted from special sources (e.g. RNA extracted from CLIP (Cross Linking and Immunoprecipitation) and from subtractive hybridization).

The key idea of the present invention is to randomly fragment a purified target sequence template to generate a pool of random, overlapping, and short sequence fragments that collectively cover the whole targeted region of interest in an unbiased way, which can be used as capture probes to capture target sequences from a nucleic acid sample.

In some embodiment of the invention, the method of selecting and enriching target sequences for a subset sequences from a nucleic acid sample comprises the following steps: a) obtaining a purified target sequence template that encompasses all the sequences of the target sequences of interest; b) preparing a library of target-capturing sequences comprising random DNA/RNA fragments generated from the target sequence template, wherein the target-capturing sequences have a capture domain; c) hybridizing said nucleic acid sample with the target-capturing sequences; d) capturing hybrids of the target-capturing sequences and the target sequences. In some embodiment, the hybrids of the target-capturing sequences and the target sequence are captured by attaching the capture domain to a functional domain immobilized on a solid surface. Once captured, target sequences can be eluted from the hybrids under appropriate conditions.

In some embodiment of the invention, the random DNA fragments are generated from the target sequence template using enzymatic methods, including, but not limited to, using a single or a combination of nucleases such as Fragmentase™ (NEB, Ipswich, Mass.), DNAse I, and Benzonase® (EMD, Gibbstown, N.J.), and other endonucleases. Fragmentase™ is an endonuclease that generates dsDNA breaks in a time-dependent manner to yield 100-800 bp DNA fragments. Benzonase® is genetically engineered endonuclease from Serratia marcescens that can effectively cleavage both DNAs and RNAs.

In one embodiment, an in vitro transposition system is used to generate random DNA fragments from the target sequences template. In an in vitro transposition reaction, a transposase and a transposon end are incubated with the target sequence template. The transposases and transposon ends form a transposome complex, which catalyze insertion of transposon ends to random or almost random locations of the target sequence template. By varying the concentration of transposome complexes and reaction time, the size distribution of the resulting DNA fragments can be optimized for capture efficiency and signal to noise ratio. In some embodiment, a capture domain is incorporated in the transposon end, for example, a biotinylated nucleotide is incorporated into the transposon ends. The biotinylated transposon ends allow capturing the target-capturing sequences and the associated target sequences by streptavidin-coated magnetic beads.

In some embodiment of the invention, a capture domain is linked to the random DNA fragments to make a target-capturing sequence pool. For example, a dsDNA adaptor sequence with a capture domain or two different adaptor sequences can be ligated to the ends of the random DNA fragments. The capture domain used herein refers to a chemical structure or moiety incorporated in a nucleic acid sequence, wherein the chemical structure or moiety comprises an affinity binding group (e.g. a biotin, an antigen, an ligand, which allows the capture of the capture domain containing nucleic acid by affinity binding to its binding partner) or a cross-linking moiety (e.g. a modified nucleotide that is capable of photochemically or chemically forming a covalent bond to substrates on a solid surfaces). The target-capturing library immobilized to a solid surface by a covalent bond allows hybridization under more stringent conditions to increase specificity and reuse of the immobilized target-capturing library.

In some embodiment, the target-capturing library first hybridizes to target sequences in solution and are later separated from the rest of the nucleic acid population via binding of the capture domain to its binding partners on a solid surface. In another embodiment, the target-capturing sequences are first immobilized to a solid support such as a magnetic bead via the capture domain. Immobilized target-capturing sequences are then hybridized with target sequences and capture the target sequences onto the solid support. As single stranded target-capturing sequences are expected to have better capturing efficiency than their double stranded counterparts. It is desirable to make single stranded DNA (ssDNA) target-capturing sequences from the double stranded DNA (dsDNA) fragments. In some embodiment, target-capturing sequences are RNA sequences, which are transcribed from dsDNA fragments above.

In some embodiment of the invention, the random DNA/RNA fragments are generated from the target sequence template using physical means, including, but not limited to, sonication, nebulization, physical shearing, and heating. The DNA fragments generated by physical means then go through a repair and end polishing process to become ligatable dsDNA sequences with blunt ends or A-overhangs, which can be ligated to a capture domain to make a target-capturing library.

In some embodiment of the invention, the target sequence template is a RNA sequence. The advantage of using RNA as a target sequence template is that the strand specific information is maintained in the single-stranded target capturing RNA or cDNA library. Similar to making target-capturing sequences from a target sequence DNA template, the target sequence RNA template needs to be broken into random fragments using enzymatic and physical means. Random RNA fragments are then linked to one or two RNA or DNA tags using ligases that can efficiently ligate RNA molecules. The tagged RNA fragments generated from the target sequence RNA template can be directly used as the target-capturing library. Alternatively, the tagged RNA fragments can be converted to a complementary DNA sequence library using a reverse transcription reaction.

In some embodiment of the invention, the random DNA/RNA fragments generated from the target sequence template are ligated to one or two sequence tags, wherein one sequence tag comprises an attachment domain that allows the random DNA/RNA fragments to be immobilized to a solid support via a non-covalent or a covalent bond. The random DNA/RNA fragments attached to the solid support can be directly used as the target-capturing library or can be used to generate target capturing sequences using DNA polymerization, RNA transcription, or reverse transcription reactions.

In some embodiment, the present invention provides a kit for selecting and enriching target sequences from a nucleic acid sample, comprising: a transposase, a transposon end incorporated with a capture domain, and a solid substance with a function domain that is capable of interacting with the capture domain. The capture domain may comprise an affinity binding group or a crosslinking moiety. The function domain on the solid phase can bind to the capture domain by affinity binding or form a covalent bond with the crosslinking moiety of the capture domain. For example, the capture domain may have a biotin moiety and the function domain on the solid substance is streptavidin. The capture domain may have a photoactivatible nucleotide analogue incorporated in a specific adapter sequence, and the function domain is a nucleic acid sequence complementary to the specific adapter sequence. The kit may further comprise a dsDNA specific exonuclease for making ssDNA from dsDNA target-capturing sequences.

In some embodiment, the present invention provides a kit for selecting and enriching target sequences from a nucleic acid sample, comprising: one or a combination of nucleases selected from DNAse I, Fragmentase™, and Bensonase®, a DNA polymerase, an adaptor sequence with a capture domain, and a solid substance with a function domain that is capable of interacting with the capture domain. The capture domain may comprise an affinity binding group or a crosslinking moiety. The function domain on the solid phase can bind to the capture domain by affinity binding or form a covalent bond with the crosslinking moiety of the capture domain. The DNA polymerase can be Taq DNA polymerase, T7 DNA polymerase, T4 DNA polymerase, or DNA polymerase I, the large fragment. The kit may further comprise a dsDNA specific exonuclease for making ssDNA from dsDNA target-capturing sequences.

### BRIEF DESCRIPTION OF THE DRAWINGS

FIG. 1. A schematic diagram showing the procedure to enrich target sequences using a target-capturing sequence library.

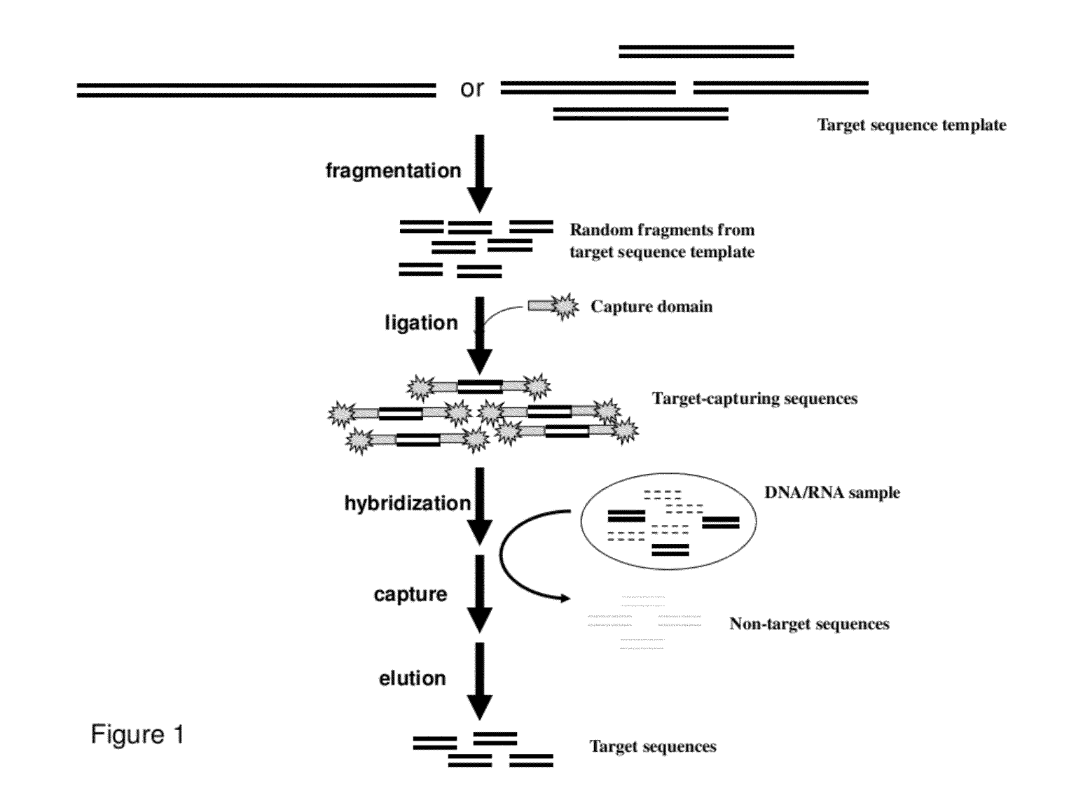

FIG. 2. A schematic diagram showing the procedure of making single stranded target-capturing beads.

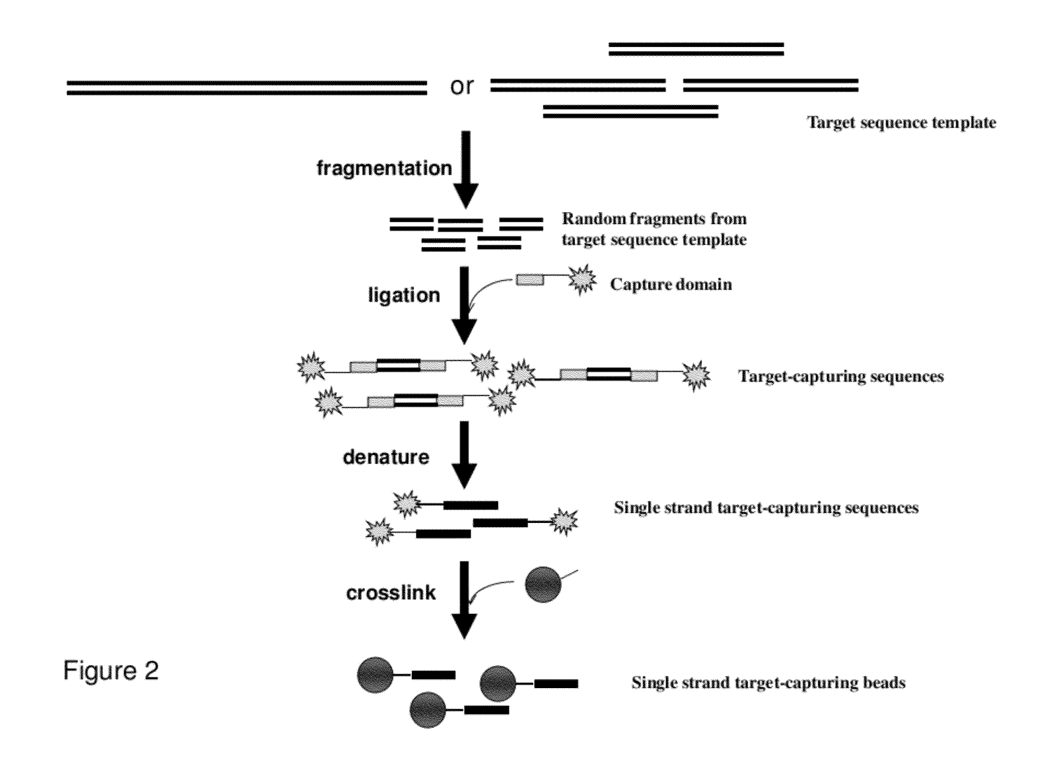

FIG. 3. A schematic diagram showing the procedure of making target-capturing beads using an in vitro transposition system.

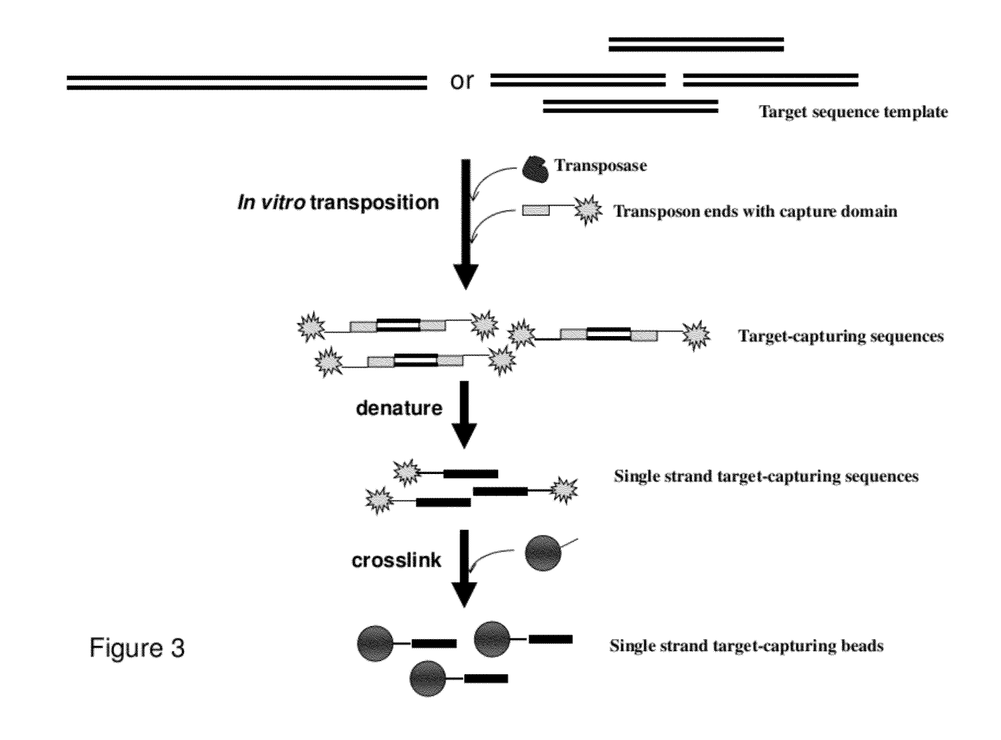

### DETAILED DESCRIPTION OF THE INVENTION

The present invention provides a method for making a targeted DNA library specific for a portion of genomic region or a subset of transcriptome using a target-capturing library. A key feature of the present invention is to use a target-capturing library of overlapping and random short DNA fragmentsrn generated from a purified target sequence template to capture target sequences in a DNA/RNA sample. The present invention provides an efficient and cost-effective target selection method ideal for targeted genome sequencing.

Comparing to the existing oligonucleotide array or PCR-based technologies for target selection, the present invention offers many advantages. Both oligonucleotide array and PCR-based target selection technologies require design and synthesis of thousands of oligonucleotides, which is costly and labor-intensive. Without the need of designed oligonucleotides, the present invention can greatly reduce the cost for targeted sample preparation. Although a good algorithm for oligonucleotide design may be able to reduce design bias and increase uniformity, it has limitations. The present invention uses a target-capturing library of overlapping and random sequences that collectively cover the whole range of target sequences to be sequenced, thus eliminating design bias and greatly increase uniformity in sequencing. In addition, the size distribution of target-capturing sequences can be controlled for optimal specificity and signal-to-noise ratio. Another advantage of the present invention is that the execution of the invention does not depend on availability of sequence information of the region of interest. Unlike the oligonucleotide and PCR-based technologies, the present invention can therefore be applied to prepare samples for genome regions or transcriptome subsets with no sequence information available.

The term “a” and “an” and “the” as used to describe the invention, should be construed to cover both the singular and the plural, unless explicitly indicated otherwise, or clearly contradicted by context.

The term “target sequences” or “target nucleic acids” as used interchangeably herein, refers to any nucleic acid sequences of interest, which are constituted of a selected subset of sequences within the whole population of sequences in a sample. The target sequences can be single-stranded or double-stranded sequences. The selected sequences of interest, for example, may be related to a single diseases, multiple diseases, an important signaling pathway, a particular genomic region, a regulatory region, a group of related genes. etc. These sequences of interest may be a subject of next generation sequencing. A target sequence can be a long stretch of genomic sequence, a cDNA sequence, or a short DNA fragment of the region of interest. The target sequences are particularly referred to a collection of short DNA sequences originated from a region of interest that are subjected to next generation sequencing. A target sequence can also be RNA sequences of interest, for example, rRNAs, mRNAs, siRNAs, snRNA, or RNAs extracted from special sources (e.g. RNA extracted from CLIP (Cross Linking and Immunoprecipitation) and from subtractive hybridization).

The term “nucleic acid sample” as used herein, refers to DNA or RNA sequences obtained from any sources, which include a mixture of sequences with target sequences and non-target sequences. For example, a nucleic acid sample may be prepared from cells, tissues, organs, any other biological and environmental sources. A nucleic acid sample may comprise whole genomic sequences, subgenomic sequences, chromosomal sequences, PCR products, cDNA sequences, mRNA sequences or whole transcriptome sequences. The target sequences of interest are only a subset of a nucleic acid sample.

The term “a target sequence template” as used herein, refers to a collection of purified DNA/RNA sequences that collectively cover the whole range or a substantial portion of all the target sequences of interest. A target sequence template does not necessarily have exactly the same sequence as target sequences. Target sequences may have sequence mutations that are different from the target sequence template (e.g. single nucleotide polymorphism). A target sequence template can be a continuous region of a DNA sequence (e.g. a BAC construct of a genomic region, or a genomic regulatory region of a gene) or a collection of DNA sequences (e.g. PCR products of genes of interest, or cDNA sequences) or DNAs extracted from special sources (e.g. DNAs from chromatin immunoprecipitation assays). For example, if genomic regions of a particular disease gene are of interest, the target sequence template can be DNA purified from a BAC construct or multiple BAC constructs encompassing genomic loci for the particular disease gene. If exon regions of a particular transcriptome are of interest, the cDNA sequences reverse transcribed from mRNAs of the particular transcriptome can be used as a target sequence template. The target sequence template can also be a pool of PCR products of genes of interest (e.g. a pool of PCR products of cancer related genes). A target sequence template can also be RNA sequences, for example, rRNAs, mRNAs, siRNAs, snRNA, or RNAs extracted from special sources (e.g. RNA extracted from CLIP (Cross Linking and Immunoprecipitation) or RNAs extracted from subtractive hybridization), which cover the sequences of interest. Target sequences isolated and purified from one source (e.g. from one patient) can act as target sequence templates to make target-capturing sequences for selecting the same target sequences from a different source (e.g. from a different patient with same disease).

The term “random DNA/RNA fragments” as used herein, refers to a portion or a segment of a larger DNA or RNA sequence that is cleaved or released from the larger DNA or RNA sequence at random or almost random locations. The collection of all the random nucleic acid fragments generated from a particular nucleic acid sequence should represent the whole sequence of the particular nucleic acid sequence in a relatively unbiased manner. The random DNA/RNA fragments particularly refer to random fragments generated from DNA/RNA target sequence templates. The process of generating smaller fragments from a larger nucleic acid sequence refers as “fragmenting”. Random DNA/RNA fragments can be generated by enzymatic or physical means.

The term “target-capturing sequences” as used herein, refers to nucleic acid sequences comprising sequences substantially complimentary to target sequences. Optionally, the target-capturing sequences have a capture domain or capable of linking to a capture domain, which allows the capture of target-capturing sequences and associated target sequences.

The term “transposon end” or “transposon end sequence” as used herein, refers to a double-stranded DNA consisting of nucleotide sequences that are necessary for forming a functional complex with a transposase that is functional in an in vitro transposition reaction. The transposon end forms a functional complex or a “transposome complex” with the transposase, which is essential for inserting the dsDNA transposon end into a target dsDNA when incubating in an in vitro transposition reaction. The transposon end have two complimentary strands of DNA sequences consisting of a first strand (a transferable strand) which can be covalently joined to 5′ of the target DNA sequence, and a second strand (a non-transferable strand) which is not directly joined to the target DNA sequence in a transposition reaction, but anneals to the first strand of the transposon end. Each transposase recognizes specific transposon end sequences. For example, bacterial transposase Tn5 recognizes a 19 base pair transposon end sequence as the following:

Transferable strand: 5′ AGATGTGTATAAGAGACAG 3′

Non-transferable strand: 5′ CTGTCTCTTATACACATCT 3′

The term “transposase' as used herein, refers to an enzyme that forms a functional complex with a transposon end sequence and catalyzes the insertion of the transposon end sequence into a target DNA sequence. Of particular interest to the present invention are hyperactive transposases such as hyperactive Tn5, Tn3 or Tn7 mutants that are capable of catalyzing in vitro transfer of transposon ends to random locations of target DNA sequences. Retroviral integrases are also included in the meaning of transposases as defined herein.

The term “transposition reaction' or “in vitro transposition reaction” as used herein, refers to a reaction that a transposase forms a complex with a transposon end and a target DNA sequence, makes a break at a random location of the target DNA, and catalyzes the transfer or transposition of the transposon end to the target DNA. When two transposon ends are transferred to the same target DNA, a DNA fragment between the adjacent insertions of two transposon ends is cleaved and separated from the target DNA. The transposition reaction can therefore be used to generate random fragments of a target DNA. Transposition creates a 9-bp single-stranded sequence immediately flanking the transposon insertion site. The mechanism of transposition reactions are well documented in the literatures, for example, in the U.S. Patent Application Publication No. 2010/012,0098 and U.S. Pat. No. 5,965,443, which is incorporated by reference herein.

The term “capture domain” as used herein, refers to a structure or a moiety incorporated into a nucleic acid sequence that allows the separation of the capture domain containing nucleic acid sequence and any specifically bound nucleic acids from the rest of nucleic acid populations. The capture domain may comprise an affinity binding group which allows the capture of the capture domain containing nucleic acid by affinity binding to its binding partner, or a cross-linking moiety that is capable of photochemically or chemically forming a covalent bond to another substrate, which can be immobilized to a solid surface.

Methods to separate nucleic acids by affinity binding are well known to those of ordinary skill in the art. Non-limiting examples of the separation methods include using physical separation, ligand-receptor binding, antigen-antibody association, or complementary nucleic acid pairing. For example, the capture domain may comprise a ligand that allows capture by ligand-receptor binding. The capture domain may be an antigen that can be separated by binding to its antibody coupled on structures such as agarose, plastic or glass beads. In another example, capture domain may comprise a specific nucleic acid sequence which can bind to its complementary nucleic acid immobilized to magnetic beads. In some embodiment of the invention, a biotin moiety is incorporated into the nucleic acid as a capture domain. The biotin-containing nucleic acids can be separated by binding to immobilized streptavidin or avidin (e.g. streptavidin-coated magnetic beads or avidin-coated magnetic beads).

The crosslinking moieties capable of forming a covalent crosslink between a nucleic acid and other substrate are well known to those skilled in the art. Examples of the crosslinking moieties suitable for DNA modification are disclosed in U.S. Pat. Nos. 4,599,303, 4,826,967, 5,082,934, and 6,005,093, which are incorporated by reference herein. The crosslinking moiety can be activated to form a covalent bond chemically or photochemically. Light-activated crosslinkers are preferable for the purpose of the current method because a crosslinking event can be stimulated at an optimal moment. The capture domain with a photoactivatible crosslinking moiety can be used to make target-capturing libraries covalently bond to solid surfaces.

The present invention provides a method for enriching and selecting DNA sequences of a targeted genome region or a subset of a transcriptome using a target-capturing DNA library. The target-capturing DNA library is generated by randomly fragmenting a target DNA template, which ideally encompasses all the DNA sequences of interest. The pool of random DNA fragments generated from the target DNA template is linked to a capture domain to make a target-capturing DNA library, which is used to capture sequences of interest from a population of nucleic acid sequences.

In some embodiment, the present invention provides a method of selecting and enriching target sequences from a nucleic acid sample, comprising: a) obtaining a purified target sequence template that encompasses all the sequences of the target sequences of interest; b) preparing a library of target-capturing sequences comprising random DNA/RNA fragments generated from a target sequence template encompassing the target sequences, wherein the target-capturing sequences have a capture domain; c) hybridizing the nucleic acid sample with the target-capturing sequences; d) capturing hybrids of the target-capturing sequences and the target sequences. In some embodiment, the hybrids of the target-capturing sequences and the target sequence are captured by attaching the capture domain to a functional domain immobilized to a solid surface. Once captured, target sequences can be eluted from the hybrids under appropriate conditions.

In some embodiment of the invention, the random DNA fragments are generated from the target sequence template using enzymatic methods. The target sequence template can be digested by a single or combination of endonucleases such as Fragmentase™, DNAse I, and Benzonase® to generate random DNA fragments with different size distributions. Benzonase® is a genetically engineered endonuclease from Serratia marcescens that can effectively make cleavage inside both DNAs and RNAs. DNA endonucleases like DNAse I and Benzonase®, when incubating with DNA at high concentrations, non-specifically cleaves DNA to release oligonucleotides 2 to 5 bases in length. By lowering enzyme concentrations and reducing incubation time, DNAse I and Benzonase® can be used to generate random DNA fragments with different sizes. Ideal length of target-capturing sequences can range from 50 to 100 bp, 100 to 150 bp, 150 to 200 bp, or 200 to 500 bp. Fragmentase™ is an endonuclease that generates dsDNA breaks in a time-dependent manner. Fragmentase™ contains two enzymes, one randomly makes a nick in a dsDNA and the other recognizes the nick site and cuts the opposite DNA strand across from the nick. Fragmentase™ can be used to generate 100-800 bp dsDNA fragments depending on reaction time. The concentration of Fragmentase™ and reaction time can be optimized for capture efficiency and high signal-to-noise ratio.

In some embodiment of the invention, an in vitro transposition system is used to generate random DNA fragments from the target sequence template. In an in vitro transposition reaction, a transposase and a transposon end are incubated with the target sequence template. The transposases and transposon ends form a transposome complex, which catalyze insertion of transposon ends to random or almost random locations of the target sequence template. By varying the concentration of transposome complexes, the size distribution of the resulting DNA fragments can be optimized for capture efficiency and signal to noise ratio. In some embodiment, a capture domain is included in a transposon end, for example, a biotinylated nucleotide is incorporated into 5′ of the transposon end. The biotinylated transposon ends allow capturing the target-capturing sequences and the associated target sequences by streptavidin-coated magnetic beads.

In some embodiment, a capture domain can be linked to the above random DNA fragments to make a target-capturing sequence pool. For example, an adapter sequence with a capture domain can be ligated to either ends of the random DNA fragments. The capture domain used herein refers to a chemical structure or moiety incorporated into a nucleic acid or an oligonucleotide sequence, wherein the chemical structure or moiety comprises an affinity binding group (e.g. a biotin, an antigen, an ligand, which allows the capture of the capture domain containing sequences by affinity binding to its binding partner) or a cross-linking moiety (e.g. a modified nucleotide that is capable of photochemically or chemically forming a covalent bond to substrates on a solid surfaces). The target-capturing library immobilized to a solid surface by a covalent bond allows hybridization under more stringent conditions to increase specificity and reuse of the immobilized target-capturing library. In some embodiment, the capture domain comprises a photoactivatible nucleotide analogue, which can form a covalent bond with nucleotides on a complimentary DNA strand upon UV light activation. Examples of photoactivatible nucleotide analogues suitable for present invention are disclosed in U.S. Pat. No. 5,082,934 by Saba et al. and Nucleic Acids Symposium Series No. 49: 57-58 (2005) by Greenberg, which is incorporated by reference herein. The photoactivatible nucleotide analogue can be incorporated into an adapter sequence that is linked to the target-capturing sequences, or the photoactivatible nucleotide can be incorporated into 5′ of the transposon ends which are linked to the target-capturing sequences in the transposition reaction. A single-stranded sequence complementary to the above adapter sequence or the 5′ of the transposon end is linked to a solid surface such as magnetic beads, thus allowing hybridization and covalently linking the target-capturing sequence to the complementary sequences on the magnetic beads. Once the target-capturing sequences are covalently bound to the magnetic beads, stringent washing conditions can be applied to rigorously remove sequences not covalently linked to the magnetic beads, thus resulting in magnetic beads covalently bound with single-stranded target-capturing sequences. In some embodiment of the invention, the adapter sequence has a 5′-single-stranded overhang sequence incorporated with a photoactivatible moiety, which is made complementary to a nucleic acid sequence attached to magnetic beads.

In some embodiment of the invention, single-stranded target-capturing sequences containing modified ends can directly form a covalent bond with reactive groups on a solid surface. A solid surface is provided by a solid support which includes, but not limited to, cellulose, Sephadex, Sephacryl, agarose, silica, polystyrene, and glass beads. A typical solid support used in the present invention is a magnetic bead which can be easily separated by magnetic fields. Methods for covalently attaching single-stranded DNA to a solid surface are well known to those skilled in the art. For example, Lund et al. (Nucleic Acids Research, 1988, Vol 16 (22): 10861-10880) described a method of carbodiimide-mediated end-attachment of 5′-NH₂ modified nucleic acid to carboxyl groups on magnetic beads. Penchovsky et al. described a light-dependent covalent immobilization of 5′-NH₂ modified nucleic acid to paramagnetic beads. These methods are suitable for the purpose of the present invention, both of which are incorporated by reference herein. An adapter dsDNA sequence with 5′-NH₂ modification is ligated to the target-capturing dsDNA fragments. The dsDNA fragments can be made single-stranded by first heating for 5 to 10 minutes in the boiling water followed by rapid cooling in ice. Single-stranded target-capturing sequence can be immobilized to magnetic beads as described by Lund and Penchovsky.

In some embodiment, the target-capturing sequences first bind to target sequences in solution and are later separated from the rest of the nucleic acid population via binding of the capture domain to its binding partner which is immobilized to a solid support. In another embodiment, the target-capturing sequences are first immobilized to a solid support such as a magnetic bead via the capture domain. Immobilized target-capturing sequences are then hybridized with target sequences and capture the target sequences onto the solid support.

As single stranded target-capturing sequences are expected to have better capturing efficiency than their double stranded counterparts. It may be desirable to make single stranded target-capturing sequences from the double stranded DNA fragments. In some embodiment, double stranded target-capturing sequences are made single stranded using a dsDNA specific exonuclease. Double stranded DNA specific exonucleases, for example, T7 exonuclease, Lambda exonuclease, and exonuclease III, that selectively digest one strand out of the two strands of a dsDNA can be used for this purpose. In one embodiment of the invention, dsDNA specific exonucleases are allowed to bind to both ends of dsDNA fragments and digest away one DNA strand either from 5′ to 3′ (e.g. Lambda exonuclease) or from 3′ to 5′ (e.g. exonuclease III). Giving enough reaction time, two non-overlapping single stranded DNAs from different strands of the parent dsDNA will be generated when two exonucleases meet each other in the middle of the parent dsDNA. A good exonuclease to be used is the one that can digest a DNA strand progressively and have high specificity towards dsDNA vs. ssDNA.

In another embodiment, only one end of the double stranded target-capturing sequence is modified to be protected from digestion by an exonuclease, which is used to selectively digest the unprotected strand. The protective modification can, for example, provide a steric hindrance to prevent exonuclease binding or remove an essential structural element required for exonuclease recognition. The asymmetric modification can be achieved either by ligating two different adaptor sequences (only one with the protective modification) to the target-capturing dsDNA, or using PCR to add two different adaptors to the target-capturing dsDNA. Different adaptor sequences can also be added to transposon ends and linked to a target-capturing dsDNA during a transposition reaction. For example, Lambda exonuclease is a highly progressive enzyme that preferably digests the 5′ phosphorylated strand of a dsDNA. Using one primer with a 5′ phosphate and another primer with a 5′-OH, target-capturing sequences can be amplified by PCR to produce sequences with a 5′ phosphate on only one of the two ends. Lambda exonuclease will selectively digest the DNA strand with a 5′ phosphate and produce a single stranded DNA with a 5′-OH. In another embodiment, the protective modification can be the same as the capture domain. For example, one primer can be modified to incorporate a biotin moiety at its 5′ or have a crosslinking moiety such as a 5′-NH₂ modification. The DNA strand with the modification/capture domain will be protected from exonuclease digestion.

Another method to generate single-stranded target-capturing DNA is to ligate two different tags to double stranded target-capturing DNAs and perform linear amplification of one strand of the dsDNAs using only one of the two tags as a primer. For each amplification cycle, a single-stranded DNA is produced and the single-stranded DNA will increase in a linear fashion. For example, with 10 amplification cycles, 90% of the target-capturing DNA will be single-stranded.

The target-capturing probes can also be RNAs instead of DNAs. The advantage of using RNA probes includes higher binding affinity of RNA/DNA hybrids and easy removal of RNA probes by RNAse digestion once target DNAs are captured. To make target-capturing RNA probes, ligate a DNA tag comprising a RNA polymerase-specific promoter (e.g. T7, T3, Sp6 promoter) sequence to one end of random DNA fragments generated from target sequence templates. The DNA fragments from target sequence templates can then be transcribed into RNA probes using in vitro transcription protocols well known to those skilled in the art. Biotin-labeled ribonucleotides, for example, Biotin-14-CTP and Bio-11-UTP (Invitrogen, Carlsbad, Calif.), can be incorporated into RNA probes to obtain biotinylated probes during in vitro transcription reactions. Or RNA primers incorporated with biotinylated nucleotides can be used to make biotinylated RNA target-capturing probes.

In some embodiment, the random DNA fragments are generated from the target sequence template using physical means, including, but not limited to, sonication, nebulization and physical shearing. The method of generating DNA fragments using sonication, nebulization, or physical shearing is well known to those skilled in the art. The DNA fragments generated by physical means then go through a repair and end polishing process to become ligatable dsDNA sequences with blunt ends or A-overhangs, which can be ligated to a capture domain containing nucleic acid sequence to make a target-capturing library.

In some embodiment of the invention, the target sequence template is a RNA sequence. One advantage of using RNA as a target sequence template is that the strand specific information is maintained in the resulting single-stranded target-capturing RNA or cDNA library. Similar to making target-capturing sequences from a target sequence DNA template, the target sequence RNA template needs to be broken into random fragments using enzymatic and physical means well known to those of ordinary skills in the art. For example, heating RNA molecules can lead to breaking RNAs into random fragments. By varying the heating time and temperature, the size range of broken RNA fragments can be controlled. Random RNA fragments are then linked to one or two RNA tags using ligases that can efficiently ligate RNA molecules. Since single-stranded RNA molecules can only ligate to sequence tags in a fixed direction, information of RNA 5′->3′ strand direction is maintained in the resulting tagged random RNA fragments. A capture domain (e.g. a biotinylated or a photoactive nucleotide moiety) can be incorporated into one of the tags and the tagged RNA fragments can be directly used as the target-capturing sequence library. Alternatively, the tagged RNA fragments can be converted to a complementary DNA target-capturing sequence library using a DNA primer complementary to one of the tags and a reverse transcriptase.

In some embodiment of the invention, the random DNA/RNA fragments generated from the target sequence template are ligated to one or two sequence tags, wherein one sequence tag comprises an attachment domain (e.g. a biotinylated nucleotide moiety or a photoactive nucleotide moiety) that allows the random DNA/RNA fragments fixed to a solid support via a non-covalent or a covalent bond. The methods to non-covalently or covalently immobilize tagged random DNA/RNA fragments to a solid support (e.g. magnetic beads) are described above. The random DNA/RNA fragments immobilized to the solid support can be used to further generate target-capturing sequences using DNA polymerization, RNA transcription, or reverse transcription reactions. If the immobilized random fragments are DNA sequences, single-stranded target-capturing DNA sequences can be generated using a DNA primer complementary to one of its sequence tags and DNA polymerase. The immobilized random DNA fragments can also be incorporated with a RNA polymerase promoter (e.g. T7, T3, or Sp6 promoter). Single-stranded target-capturing RNA sequences can be then generated using a RNA primer and a RNA polymerase. dsDNA can also be generated from double tagged, immobilized random DNA fragments using two DNA primers and a polymerase chain reaction. If the immobilized random fragments are RNA sequences, complementary DNA sequences can be generated from those RNA sequences via a reverse transcription reaction well known to those skilled of the art.

In some embodiment, the present invention provide a kit for selecting and enriching target sequences from a DNA sample, comprising: a transposase, a transposon end incorporated with a capture domain, and a solid substance with a function domain that is capable of interacting with the capture domain. The capture domain may comprise an affinity binding group or a crosslinking moiety. The function domain on the solid phase can bind to the capture domain by affinity binding or form a covalent bond with the crosslinking moiety of the capture domain. For example, the capture domain may have a biotin moiety and the function domain on the solid substance is streptavidin. The capture domain may have a photoactivatible nucleotide analogue incorporated in a specific adapter sequence, and the function domain is a nucleic acid sequence complementary to the specific adapter sequence. In some embodiment, the kit further comprises a dsDNA specific exonuclease for generating single stranded target-capturing DNA.

In some embodiment, the present invention provides a kit for selecting and enriching target sequences from a nucleic acid sample, comprising: one or a combination of nucleases, a DNA polymerase, an adaptor sequence with a capture domain, and a solid substance with a function domain that is capable of interacting with the capture domain. The nucleases including, but not limited to, DNAse I, Fragmentase™, Bensonase®, are used individually or in combination to fragment target sequence templates into random DNA fragments. The capture domain may comprise an affinity binding group or a crosslinking moiety. The function domain on the solid phase can bind to the capture domain by affinity binding or form a covalent bond with the crosslinking moiety of the capture domain. The DNA polymerase, which is used to fill in 5′ overhangs and chew back 3′ overhangs, can be Taq DNA polymerase, T7 DNA polymerase, T4 DNA polymerase, or DNA polymerase I, the large fragment. The kit may further comprise a dsDNA specific exonuclease for making ssDNA from dsDNA target-capturing sequences.

### EXAMPLES

### Example 1

### Procedure for Making a Biotin-Labeled Target-Capturing Sequence Library

Starting materials that can be used as target sequence templates for generating a target-capturing library include, but not limited to, commercially available large genomic DNA fragments such as BAC clones, or a collection of PCR fragments generated from amplification of areas of interest, or collection of cDNA clones from commercial source or private collections, or areas of genomes/transcriptomes amplified through rolling circle amplification.

Relatively large amounts of target sequence template DNA are needed to generate target-capturing libaries for extended use. Large quantity materials that commercially available are often preferred for its reproducibility and cost effectiveness. Amplified materials are often recommended to be produced in large batches to sustain consistency.

Target sequence templates are fragmented into desired sizes by incubating with an EZ-Tn5™ transposase (EpiCentre BioTechnologies, Madison, Wis.) and a transposon end sequence specific for Tn5 transposase. EZ-Tn5™ transposase is a hyperactive mutant of Tn5 with three point mutations at the aa₅₄, aa₅₆, and aa₃₇₂ of the wild-type Tn5 transposase. A 5′ biotinylated nucleotide is incorporated into the transposon end sequence. The working conditions for this EZ-Tn5 transposase based in vitro transposition reaction were described in U.S. Patent Application Publication No. 2010/0120098 and U.S. Pat. No. 5,965,443, which are incorporated by reference herein. Generally, target DNA is incubated with EZ-Tn5™ transposase and a specific transposon end sequence in the transposition reaction buffer. The amount of target DNA, EZ-Tn5™, and the specific transposon end sequence may vary depending on the application. Buffer components and concentration, and incubation temperature and time may vary according to desired fragment size distribution. The reaction can be stopped by adding a stop solution (10% sucrose, 66 mM EDTA, 20 mM TRIS, 0.1% SDS, 0.9% Orange G, and 100 μg/ml Protease K) and heating at 50° C. for 10 minutes. The DNA fragment size distribution can be checked on a 1% agarose gel. The transposition reaction will fragment the target sequence template and add Biotin-labeled transposon ends to the DNA fragments.

Once the transposition reaction is completed, Biotin-labeled DNA fragments will be purified using Zymo DNA Clean and Concentrator kit and serve as the target-capturing library. The Biotin-labeled target-capturing library is now ready to be used for hybridization and capture of target sequences of interest.

Incubate Biotin-labeled target-capturing library and a DNA sample with target sequences of interest under appropriate condition so that the target-capturing sequences will specifically hybridize with the target sequences. After reaching the equilibrium of the hybridization, the hybrids of biotin-labeled target-capturing sequences and target sequences are cooled down to room temperature and captured by streptavidin-coated magnetic beads, Dynabeads (Life Technologies, Carslbad, Calif.) using a magnetic field according to manufacture's instruction.

### Example 2

### Procedure for Making Target-Capturing Beads Using Photoactivation

This example illustrate the procedure for making target-capturing beads using photoactivation. A single-stranded adaptor sequence incorporated with a photoactivatible nucleotide analogue is attached as a 5′ overhang to the dsDNA transposon end sequence. The photoactivatible nucleotide analogues disclosed in U.S. Pat. No. 5,082,934 that can form a covalent bond with nucleotides on the complementary strand upon activation by UV radiation can be used for the purpose of the present invention. Single stranded sequences that are complimentary to the adaptor sequence are chemically synthesized and attached to solid capture beads.

Target-capturing sequence library is generated using a transposition reaction as described in Example 1, with a transposon end sequence incorporated with a photoactivatible nucleotide analogue. Incubate the target-capturing sequences with photoactivatible adaptor sequences and the solid capture beads under conditions that allow specific hybridization of the adaptor sequences. Once the hybridization equilibrium is reached, the photoactivatible nucleotide analogue of the target-capture sequences can form a crosslink with the capture beads upon UV light activation. After the covalent bond is formed, stringent washing condition is applied to remove all the nucleic acid sequences that are not covalently bound to the solid capture beads. The target-capturing sequences are thus attached to the capture beads and can be used for direct capture of target sequences.

### Example 3

### Procedure for Making Target-Capturing Beads Using Chemical Crosslinking

This example illustrates the procedure of using endonuclease like DNAse I and chemical crosslinking reagents to make target-capturing beads with single-stranded sequences.

DNAse I causes random double stranded scission of DNA in the presence of Mn²⁺. The DNA fragment size can be controlled by varying the enzyme concentration, incubation time and/or temperature. To find conditions that produce desired fragment sizes, fixed amounts of DNA are incubated with different dilutions of DNAase I in Tris buffer (50 mM Tris-HCl, pH 7.5, 50 μg BSA/ml) with 10 mM Mn²⁺. The digestion can be performed at room temperature or 37° C. for different time periods and the resulting fragments are analyzed by agarose gel electrophoresis. The ideal length of DNA fragments is between 100 to 200 bp. The DNAase digestion is stopped by adding EDTA stop solution and heated at 65° C. for 5 to 10 minutes. Once an optimal condition is determined, target sequence template DNA is incubated with DNAse I under the optimal condition to produce target-capturing DNA fragments with desired sizes.

Target-capturing DNA fragments are purified using Zymo DNA Clean and Concentrator Kit (Zymo Research Corporation, Irvine, Calif.). Since DNAse I cleaves DNA at approximately the same site to produce DNA fragments with blunt ends or protruding termini with one or two nucleotide in length, the resulting DNA fragments need to be further treated to become ligatable. Incubate Target-capturing DNA fragments with T4 DNA polymerase I to fill in a 5′ extension and to chew back a 3′ extension. The enzyme reaction is stopped by heating at 70° C. and target-capturing DNA fragments are purified using a Zymo DNA Clean and Concentrator Kit.

A dsDNA adaptor sequence with NH₂ modification at 5′ end is synthesized according to a method described by Chu et al. (Chu, B. C. F. and Orgel, L. E. 1985, DNA, 4:327-331) The 5′-NH₂ modified adaptor sequence is ligated to target-capturing dsDNA fragments using T4 DNA ligase. The 5′-NH₂ modified double stranded DNA is made single stranded by first incubating in boiling water for 5 minutes followed by rapidly cooling in the ice. The 5′-NH₂ modified target-capturing ssDNAs are then covalently linked to magnetic beads with carboxyl groups in the presence of carbodiimide according to a method described by Lund et al. (Nucleic Acids Research, 1988, Vol 16 (22): 10861-10880). After the crosslinking reaction is complete, DNA fragments that are not covalently linked to the magnetic beads can be washed away under rigorous conditions (e.g. washing solution with 50% formamide, 6M urea, or 6M guanidine HCl). Single stranded target-capturing beads are thus obtained.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

What is claimed is:

1. A method for selecting and enriching target sequences from a nucleic acid sample, comprising steps of:
a, obtaining a target sequence template that encompasses sequences of said target sequences;
b, preparing a library of target-capturing sequences comprising random DNA/RNA fragments generated from said target sequence template, wherein said target-capturing sequences have a capture domain;
c, hybridizing said nucleic acid sample with said target-capturing sequences;
d, capturing hybrids of said target-capturing sequences and said target sequences.

2. The method of claim 1, wherein said target-capturing sequences are made single-stranded by removing one strand from double stranded sequences.

3. The method of claim 2, wherein a double stranded DNA specific exonuclease is used to digest one strand from double stranded DNA sequences.

4. The method of claim 3, wherein said double stranded DNA specific exonuclease is selected from lambda exonuclease, T7 exonuclease, and exonulease III.

5. The method of claim 1, wherein said target-capturing sequences are made single-stranded by selectively amplifying one strand of double-stranded DNA sequences.

6. The method of claim 1, wherein said target-capturing sequences are RNA sequences which are transcribed from said random DNA fragments generated from said target sequence template.

7. The method of claim 6, wherein said target-capturing RNA sequences are biotinylated.

8. The method of claim 1, wherein said random DNA/RNA fragments are generated from said target sequence template using an enzymatic or a physical method.

9. The method of claim 1, wherein said random DNA fragments are generated from said target sequence template using a single or a combination of endonucleases.

10. The method of claim 1, wherein said random DNA fragments are generated from said target sequence template using a transposase and a transposon end.

11. The method of claim 10, wherein said transposon end has a capture domain.

12. The method of claim 1, wherein said capture domain comprises a biotinylated nucleotide.

13. The method of claim 1, wherein said capture domain comprises a crosslinking moiety.

14. The method of claim 14, wherein said crosslinking moiety is photoactivatible.

15. The method of claim 14, wherein said crosslinking moiety is a photoactivatible nucleotide derivative.

16. The method of claim 8, wherein said physical method is selected from sonication, nebulization, physical shearing, and heating.

17. The method of claim 1, wherein said random DNA/RNA fragments generated from said target sequence template are linked to one or two sequence tags and fixed to a solid support, and wherein said target-capturing sequences are generated from said random DNA/RNA fragments fixed to said solid support.

18. The method of claim 2, wherein single-stranded target-capturing DNA sequences are generated from said fixed random DNA fragments using a DNA polymerization reaction.

19. The method of claim 2, wherein single-stranded target-capturing RNA sequences are generated from said fixed random DNA fragments using a RNA transcription reaction.

20. The method of claim 2, wherein single-stranded target-capturing DNA sequences are generated from said fixed random RNA fragments using a reverse transcription reaction.

21. A kit for selecting and enriching target sequences from a nucleic acid sample, comprising:
a, a transposase;
b, a transposon end incorporated with a capture domain;
c, a solid substance with a function domain that is capable of interacting with the capture domain;
d, optionally, a double stranded DNA specific exonuclease.

22. The kit of claim 19, wherein said capture domain is selected from a biotin moiety, a photoactivatible nucleotide analogue, and a 5′-NH₂ modified nucleotide analogue.

23. A kit for selecting and enriching target sequences from a nucleic acid sample, comprising:
a, one or a combination of nucleases selected from DNAse I, Fragmentase™, and Benzonase®
b, a DNA polymerase selected from Taq DNA polymerase, T7 DNA polymerase, T4 DNA polymerase, and DNA polymerase I, the large fragment
c, an adaptor sequence with a capture domain
d, a solid substance with a function domain that is capable of interacting with said capture domain
e, optionally, a double stranded DNA specific exonuclease
